(19) United States
(12) Patent Application Publication
(10) Pub. No.: US 2021/0030726 A1
(43) Pub. Date: Feb. 4, 2021

(54) RAPAMYCIN ANALOG FOR PREVENTION AND/OR TREATMENT OF NEURODEGNERATIVE CONDITIONS

(71) Applicant: MOUNT TAM THERAPEUTICS, INC., Burlington, VT (US)

(72) Inventors: Timothy S Powers, Sausalito, CA (US); Bryan F Cox, Spring Grove, IL (US); Richard B Marshak, Cornwall, VT (US)

(73) Assignee: Torcept Therapeutics, Inc., Middlebury, VT (US)

(21) Appl. No.: 16/968,214

(22) PCT Filed: Feb. 8, 2019
(86) PCT No.: PCT/US2019/017339

§ 371 Date: Aug. 7, 2020

Related U.S. Application Data
(60) Provisional application No. 62/628,493, filed on Feb. 9, 2018.

(51) Int. Cl. A61K 31/436; A61K 45/06; A61P 25/28

(52) U.S. Cl. CPC A61K 31/436; A61P 25/28; A61K 45/06

(57) ABSTRACT

The present invention relates to a novel rapamycin analogue of Formula (I), mixtures, methods for its production, and its use in preventing and/or treating a neurodegenerative condition.

### RELATED APPLICATIONS

This application claims priority to U.S. Ser. No. 62/628,493 filed on Feb. 9, 2018.

### BACKGROUND OF THE DISCLOSURE

Rapamycin (sirolimus) is a polyketide that is used to coat coronary stents and prevent organ transplant rejection. The art also suggests that rapamycin and rapamycin analogs can be used to treat lymphangioleiomyomatosis, pulmonary inflammation (U.S. Pat. No. 5,080,999), insulin dependent diabetes (U.S. Pat. No. 5,362,718 citing Fifth Int. Conf. Inflamm. Res. Assoc. 121 (Abstract), (1990)), certain coronary diseases (Morris, (1992) Heart Lung Transplant 11:197), leukemia and lymphoma (European Patent Application 0 525 960), and ocular inflammation (European Patent Application 0 532 862).

Rapamycin is produced by Streptomyces hygroscopicus NRRL 5491 (Sehgal et al., 1975; Vezina et al., 1975; U.S. Pat. Nos. 3,929,992; 3,993,749). For the purpose of this disclosure, rapamycin is described by the numbering convention of McAlpine et al. (1991) (see FIG. 1) in preference to the numbering conventions of Findlay et al. (1980) or Chemical Abstracts (11th Cumulative Index, 1982-1986 p60719CS). U.S. Pat. No. 5,362,718 discloses acylated prodrugs of rapamycin.

Rapamycin and its commercially available analogs Temsirolimus and Everolimus inhibit activation of T cells and B cells by binding to mTOR which, among other things, reduces the production of interleukin-2. mTOR is the catalytic subunit of two structurally distinct complexes: mTORC1 and mTORC2 (Wang et al. (2006) Journal of Biological Chemistry, 281: 24293-303). mTORC1 and mTORC2 localize to different subcellular compartments, which affects their activation and function.

Scientific evidence suggests that mTORC1 functions as a sensor of cellular nutritional and energy status and has a role in the regulation of protein synthesis (Hay et al. (2004) Genes & Development 18: 1926-45; Kim et al. (2002) Cell, 110: 163-75). The activity of mTORC1 is regulated by rapamycin analogs, insulin, growth factors, phosphatidic acid, some amino acids and amino acid derivatives, mechanical stimuli, and oxidative stress.

Scientific evidence suggests that mTORC2 functions an important regulator of the actin cytoskeleton through its stimulation of F-actin stress fibers, paxillin, RhoA, Rac1, Cdc42, and protein kinase Cα (Sarbassov et al. (2004) Current Biology 14:1296-302). mTORC2 also affecting metabolism and survival apparently through phosphorylation of Akt/PKB (Betz et al. (2013) PNAS 110: 12526-34). Akt phosphorylation by mTORC2 interacts with PDK1 and leads to full Akt activation (Sarbassov et al. (2005) Science 307: 1098-101; Stephens et al. (1998) Science 279: 710-4). In addition, mTORC2 is capable of activating IGF-IR and InsR (Yin et al. (2016) Cell Research 26: 46-65).

Rapamycin analogs (including Rapamycin) have significant therapeutic value (Huang et al, 2003). These polyketides are a potent inhibitor of the mammalian target of rapamycin (mTOR), a serine-threonine kinase downstream of the phosphatidylinositol 3-kinase (PI3K)/Akt (protein kinase B) signaling pathway that mediates cell survival and proliferation. This inhibitory activity is gained after rapamycin binds to the immunophilin FK506 binding protein 12 (FKBP12) (Dumont, F. J. and Q. X. Su, 1995). In T cells, rapamycin inhibits signaling from the IL-2 receptor and subsequent autoproliferation of the T cells resulting in immunosuppression. Rapamycin is marketed as an immunosuppressant for the treatment of organ transplant patients to prevent graft rejection (Huang et al, 2003). In addition to immunosuppression, rapamycin has found therapeutic application in cancer (Vignot et al, 2005).

Rapamycin and many rapamycin analogs have disadvantages including inducement of hyperlipidemia, cellular efflux mediated by P-glycoprotein (“P-gp”; LaPlante et al, 2002, Crowe et al, 1999) and other efflux mechanisms which pumps the compound out of cells and tends to decrease effectiveness of administered drug compound and presents challenges to the treatment of multidrug resistant cancer. Hepatic first pass loss of rapamycin is also high, which contributes further to its low oral bioavailability. The low oral bioavailability of rapamycin causes significant inter-individual variability resulting in inconsistent therapeutic outcome and difficulty in clinical management (Kuhn et al, 2001, Crowe et al, 1999).

A wide range of synthesized rapamycin analogues using the chemically available sites of the molecule are known in the art. Chemically available sites on the molecule for derivatization or replacement are known in the art to include, for example, C40 and C28 hydroxyl groups (e.g. U.S. Pat. Nos. 5,665,772; 5,362,718); C39 and C16 methoxy groups (e.g. WO 96/41807; U.S. Pat. No. 5,728,710); C32, C26 and C9 keto groups (e.g. U.S. Pat. Nos. 5,378,836; 5,138,051; 5,665,772); hydrogenation at C17, C19 and/or C21 (e.g. U.S. Pat. Nos. 5,391,730; 5,023,262); and/or the formation of oximes at C32, C40 and/or C28, (e.g., U.S. Pat. Nos. 5,563,145, 5,446,048). Analogues exhibiting resistance to metabolic attack (e.g. U.S. Pat. No. 5,912,253); bioavailability (e.g. U.S. Pat. Nos. 5,221,670; 5,955,457; WO 98/04279); and/or the production of prodrugs (e.g. U.S. Pat. Nos. 6,015,815; 5,432,183) have also been developed. Thus, it is understood in the art that the number of pharmaceutically useful and interesting analogs of rapamycin is very high and difficult to quantify.

### SUMMARY OF THE DISCLOSURE

In some embodiments, this disclosure provides a polyketide of Formula I that has an unexpected and beneficial pharmaceutical uses such as in the prevention and/or treatment of neurodegenerative conditions. In some embodiments, this disclosure provides compositions comprising the polyketide of Formula I for use in preventing and/or treating neurodegenerative conditions. In some embodiments, this disclosure provides methods of preventing and/or treating neurodegenerative conditions in a mammal in need thereof comprising administration of the polyketide of Formula I and/or compositions and/or mixtures comprising the same. Other embodiments are also contemplated as described herein and/or as may be ascertained by those of ordinary skill in the art.

### BRIEF DESCRIPTION OF THE DRAWINGS

FIG. 1: The rapamycin numbering schema used in this document.

FIG. 2. Effect of the compound of Formula I on contralateral touches in mice. FIG. 2A. Image of mice within testing device. FIG. 2B. Percent total touches. FIG. 2C. Percent total touches (3 mg/kg).

FIG. 3. Effect of the compound of Formula I on integrated density in striatum. FIG. 3A. Image analysis. FIG. 3B. Percent of control side.

FIG. 4. Effect of the compound of Formula I on TH⁺ neuronal loss. FIG. 4A. Image analysis. FIG. 4B. TH⁺ neuronal loss as percent of control side. FIG. 4C. TH staining. FIG. 4D. TH levels in SN (%). FIG. 4E. TH staining. FIG. 4F. TH levels in CPU (%).

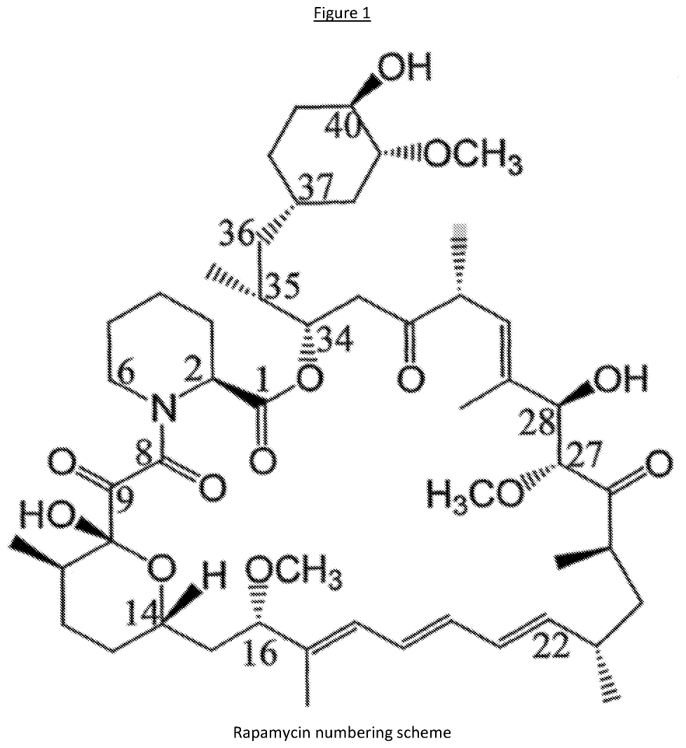

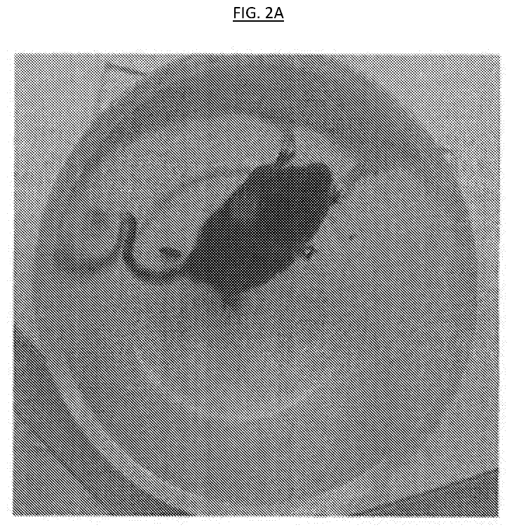

### DETAILED DESCRIPTION

This disclosure provides the polyketides of Formula I (37R-hydroxynorbornylrapamycin), compositions comprising the same, and the use of the same in preventing and/or treating neurodegenerative conditions. Compounds of Formula I have surprising and unexpectedly beneficial properties for the prevention and/or treatment of neurodegenerative conditions. The compound of Formula I is shown below:

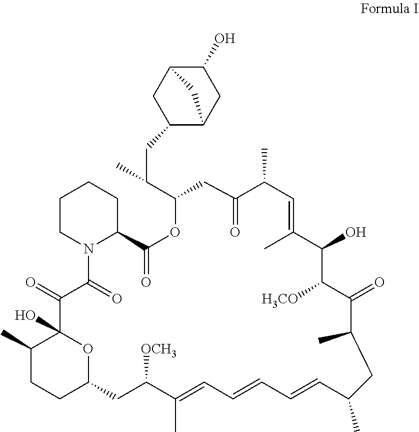 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2C[C@@H]3C[C@H]2C[C@H]3O)CC(=O)[C@H](C)/C=C(\C)[C@@H](O)[C@@H](OC)C(=O)[C@H](C)C[C@H](C)/C=C/C=C/C=C/1C]

The compound of Formula I (37R-hydroxynorbornylrapamycin) is also described in U.S. Pat. No. 9,382,266 B2, which is hereby incorporated by reference in its entirety into this disclosure.

As described herein, the compound of Formula I can be used for the prevention and/or treatment of various neurodegenerative conditions (e.g., a neurodegenerative disease), such as those resulting from the progressive loss of structure or function of neurons, including the death thereof. Exemplary neurodegenerative conditions (e.g., a neurodegenerative disease) include, but are not limited to, Alper's disease, Alzheimer's disease, amyotrophic lateral sclerosis (ALS) (motor neurone disease (MND), Lou Gehrig's Disease), Batten disease, choreic syndrome, dystonic syndrome, Friedrich's ataxia, Huntington's disease, Lewy body disease, primary or secondary Parkinson's disease, prion disease, spinocerebellar ataxia, spinal muscular dystrophy, in mammals (including human). Other conditions that can lead to or result from neurodegeneration and may benefit from administration of the compound of Formula I, and compositions comprising the same) can include, for example, aging, alcoholism, cancer, injury, stroke, infectious disease (e.g., prion disease), psychological disorders, and/or toxicity (e.g., brain iron accumulation). Symptoms of neudegeneration, such as Parkinson's disease, can include one or more of bradykinesia, or slowness in voluntary movement, which produces difficulty initiating movement as well as difficulty completing movement once it is in progress, tremors in the hands, fingers, forearm, or foot, muscle rigidity, stiff muscles, muscle pain, an expressionless, mask-like face, poor balance, and/or falls. Other neurodegenerative conditions (e.g., a neurodegenerative disease) have symptoms well-known to those of skill in the art, and may be prevented, treated and/or ameliorated by administration the compounds described herein (e.g., a compound of Formula I, and/or compositions comprising the same). Administration of a therapeutically effective amount of these compounds may improve any one or more of such symptoms following administration of a therapeutically effective amount thereof to a mammal exhibiting the same. The efficacy of the compound of Formula I and compositions comprising the same (e.g., to determine a therapeutically effective amount) may be tested in in vitro or in vivo model systems for neurodegenerative conditions (e.g., a neurodegenerative disease) which are described herein and/or are known to those skilled in the art. Such models include, but are not limited to, in vivo models for Alzheimer's disease such as using animals that express human familial Alzheimer's disease (FAD) p-amyloid precursor (APP), animals that overexpress human wild-type APP, animals that overexpress p-amyloid 1-42 (pA), animals that express FAD presenillin-1 (PS-1) (e. g. German and Eisch, 2004). An exemplary in vivo model for multiple sclerosis is the experimental autoimmune encephalomyelitis (EAE) model (see Bradl, 2003). Exemplary in vivo models for Parkinson's disease include the 1-methyl-4-phenyl-1,2,3,6-tetrahydropyridine (MPTP) model and/or the 6-hydroxydopamine (6-OHDA) model (see e.g. Emborg, 2004; Schober A. 2004). An exemplary in vivo models for Huntington's disease include the R6 model generated by the introduction of exon 1 of the human Huntington's disease (HD) gene carrying highly expanded CAG repeats into the mouse germ line (Sathasivam et al, 1999; see also Hersch and Ferrante, 2004). Other exemplary models for testing the efficacy of the compound of Formula I, and/or compositions comprising the same, are also available to those of skill in the art, and may also or alternatively be suitable for determining the efficacy (e.g., to determine a therapeutic effective amount) of the compound of Formula I.

The compound of Formula I and/or compositions comprising the same can be administered (e.g., to mammal) via any suitable route or means including, but not limited to, parenterally, orally, topically (including buccal, sublingual, or transdermally), via a medical device such as a stent, by inhalation, or via injection (e.g., subcutaneously, intramuscularly, or intravenously). The treatment optionally consists of a single dose, but preferably in many embodiments is a multiplicity of administrations over time, at the same or different dosages. The skilled artisan will recognize that the optimal quantity and spacing of individual dosages of a compound of Formula I will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

In some embodiments, the compound of Formula I or a composition comprising the same is administered as the sole active pharmaceutical agent for preventing and/or treating neurodegenerative conditions. Thus, in some embodiments, the compound of Formula I or a composition comprising the same is administered to a mammal such as a human being to prevent and/or treat a neurodegenerative condition as a pharmaceutical composition that optionally contains one or more pharmaceutical excipients, but no other active agent(s). In some embodiments, the compound of Formula I or a composition comprising the same is administered to a mammal such as a human being to prevent and/or treat a neurological condition as a pharmaceutical composition comprising at least one other active agent, and optionally also contains one or more pharmaceutical excipients. In some embodiments, the compound of Formula I or a composition comprising the same is administered to a mammal such as a human being to prevent and/or treat a neurological condition as a pharmaceutical composition optionally comprising at least one other active agent, and optionally also containing one or more pharmaceutical excipients, with at least one other composition also comprising at least one other active agent and optionally also containing one or more pharmaceutical excipients (e.g., two compositions, one containing at least the compound of Formula I and the other composition comprising at least one other active agent). Multiple compositions, each comprising one or more active agents (one of such compositions comprising the compound of Formula I), may also be administered to prevent and/or treat neurodegenerative disease. Such active agents and/or compositions, or some combination thereof, may be administered simultaneously or sequentially, and/or may be administered at the same or different sites on the mammal, and/or through the same or different routes of administration.

Active agents that may be administered to a mammal in order to prevent and/or treat a neurodegenerative condition along with the compound of Formula I, or a composition comprising the same, include but are not limited to one or more analgesics, anti-amyloidogenic, anti-cancer agents, anti-cholinergic agents, anti-convulsives, anti-depressants, anti-oxidants, anti-psychotics, anxiolytics, cell cycle inhibitors, dopamine releasing agents, dopamine replenishing agents, dopaminergic agonists, immunomodulator (e.g., anti-inflammatory, immunostimulant), metabolic modulator, neuroprotectants, nootropic agents, vasoprotectants (e.g., vasodilatory agents), and the like. Exemplary active agents that may be administered with along with the compounds described herein (e.g., a compound of Formula I) may include, for instance, any one or more of levodopa, carbidopa (SINEMET®), benserazide (MADOPAR®, MADOPAR-HBS), biperiden, catechol-O-methyltransferase (COMT) inhibitors (e.g., tolcapone, entacapone), dopamine agonists (e.g. bromocriptine, pergolide, ropinirole, pramipexole, lisuride, cabergoline, apomorphine, sumanirole, rotigotine, talipexole and dihydroergocriptine), selegiline, trihexyphenidyl, a vaccine, and the like, and/or combinations and/or mixtures thereof, optionally along with any other active agents described herein or that may be otherwise available to those of skill in the art. The compound of Formula I or a composition comprising the same may also be administered in conjunction with any one or more of surgery, gene therapy, and the like, and/or any combinations thereof, optionally along with any of the active agents and/or procedures described herein or that may be otherwise available to those of skill in the art.

The polyketide of Formula I may optionally be, but need not be, pure to be effective at preventing and/or treating neurodegenerative conditions. Thus, the polyketide of Formula I can be present in mixtures in which essentially all of the polyketide in the mixture is the polyketide of Formula I, in which 99.9% by weight of the polyketide in the mixture is the polyketide of Formula I, in which 99.5% of the polyketide in the mixture is the polyketide of Formula I, in which at least 99% of the polyketide in the mixture is the polyketide of Formula I, in which 98% of the polyketide in the mixture is the polyketide of Formula I, in which at least 95% of the polyketide in the mixture is the polyketide of Formula I, in which at least 90% of the polyketide in the mixture is the polyketide of Formula I, in which 80% of the polyketide in the mixture is the polyketide of Formula I, or in which the 70% of the polyketide in the mixture is the polyketide of Formula I.

Furthermore, in each of the foregoing aspects the compound of Formula I is optionally provided as a salt, a solvate, or an ester of the compound of Formula I. Pharmaceutically acceptable salts of the compound of formula I may include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmitic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, optionally can be useful in the preparation of salts useful as intermediates in obtaining the compounds of Formula I and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts. In an aspect of the present invention, pharmaceutically acceptable salts of the polyketide of Formula I are combined together with one or more pharmaceutically acceptable excipients, diluents, or carriers. Similarly, the polyketides of Formula I, and/or pharmaceutically acceptable salts thereof, optionally can be solvates, including alcoholic solvates and hydrates.

The compound of Formula I can be provided in a pure form, for example, in a crystalline or powdered form, and/or diluted in at least one pharmaceutically acceptable buffer, carrier, or excipient. Pharmaceutically acceptable buffers, carriers and excipients in the context of the present invention preferably do not adversely interact with the polyketide of Formula I, provide for stable formulations for suitable time periods, and are not unduly deleterious to most recipients thereof.

In some embodiments, solutions or suspensions of a compound of Formula I, and compositions comprising the same, also contain excipients such as, e.g., N,N-dimethylacetamide, dispersants e.g. polysorbate 80, surfactants, and solubilizers, e.g. polyethylene glycol, and/or Phosal 50 PG (which consists of phosphatidylcholine, soya-fatty acids, ethanol, mono/diglycerides, propylene glycol and ascorbyl palmitate).

Tablets containing the compound of Formula I optionally contain excipients such as microcrystalline cellulose, lactose (e.g. lactose monohydrate or lactose anhydrous), sodium citrate, calcium carbonate, dibasic calcium phosphate and glycine, butylated hydroxytoluene (E321), crospovidone, hypromellose, disintegrants such as starch (preferably corn, potato or tapioca starch), sodium starch glycollate, croscarmellose sodium, and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, and talc are optionally included.

Solid compositions of a similar type can also be employed as fillers in gelatin capsules. Preferred excipients in this regard include lactose, starch, a cellulose, milk sugar or high molecular weight polyethylene glycols. For aqueous suspensions and/or elixirs, the compounds of Formula I can be combined with various sweetening or flavoring agents, coloring matter or dyes, with emulsifying and/or suspending agents and with diluents such as water, ethanol, propylene glycol and glycerin, and combinations thereof.

A tablet can be made by compression or molding, optionally with one or more accessory ingredients. Compressed tablets can be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Molded tablets can be made by molding in a suitable machine a mixture of the powdered compound moistened with an inert liquid diluent. The tablets can optionally be coated or scored and can be formulated so as to provide slow or controlled release of the active ingredient therein using, for example, hydroxypropylmethylcellulose in varying proportions to provide desired release profile.

For convenience, the formulations are optionally presented in unit dosage form and can be prepared by any of the methods well known in the art of pharmacy. Such methods include the step of bringing into association the active ingredient (compound of Formula I) with the carrier which constitutes one or more accessory ingredients. In general, the formulations are prepared by uniformly and intimately bringing into association the active ingredient with liquid carriers or finely divided solid carriers or both, and then, if necessary, shaping the product.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oeyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions, the active ingredient can be dissolved in water for injection and filter sterilized before filling into a suitable vial or ampoule and sealing.

Advantageously, agents such as local anesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilized by exposure to ethylene oxide before suspending in the sterile vehicle. Advantageously, a surfactant or wetting agent is included in the composition to facilitate uniform distribution of the active ingredient.

The compound of Formula I or compositions comprising the same may also be administered using medical devices known in the art. For example, in one embodiment, a pharmaceutical composition described herein can be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Useful examples of well-known implants and modules include but are not limited to U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. In a specific embodiment, the polyketides (e.g., of Formula I or II) and compositions comprising the same may be administered using a drug-eluting stent, for example, such as one corresponding to those described in WO 01/87263 and related publications or those described by Perin (Perin, E C, 2005). Many other such implants, delivery systems, and modules are known to those skilled in the art.

The polyketides and compositions described herein comprising a polyketide of Formula I can be administered to treat, prevent, or mitigate a neurodegenerative condition (e.g., disease or other neurodegenerative medical condition) in a mammal in need thereof. In a preferred embodiment, the mammal is a human. Any appropriate neurodegenerative condition of the mammal can be treated by administering a pharmaceutically-appropriate quantity of the compound of Formula I to a mammal in need thereof. An ordinarily skill artisan can readily select the route of administration of the polyketide of Formula I as well as the quantity following routine studies, guidelines and procedures. The dosage to be administered of a compound of Formula I, or a composition comprising the same, will vary according to the particular compound, the neurodegenerative condition involved, the subject, and the nature and severity of the neurodegenerative condition, the physical condition of the subject, and the selected route of administration. The appropriate dosage can be readily determined by a person skilled in the art. For example, without limitation, a dose of about 0.1 mg up to 100 mg daily, and optionally about 0.1 to 15 mg daily (or a higher dose given less frequently) may be contemplated. In some embodiments, a dose of about 1 or 3 mg/kg of Formula I i.v. or 1, 5 or 10 mg/kg of the compound of Formula I p.o., or a dose of from 1-20 mg/kg (e.g., about any of 1, 2, 3, 4, 5, 6, 7, 8, 9, 10, 11, 12, 13, 14, 15, 17, 18, 19 or 20 mg/kg) can be administered one or more times.

The compositions may contain any suitable combination of the compound of Formula I and other components. In some preferred embodiments, the compositions of the invention contain from 0.1 weight % to 70 weight % of a compound of Formula I, preferably from 5-60 weight %, more preferably from 10 to 30 weight %, of a compound of Formula I, depending on the method of administration and other factors.

The compounds of Formula I can be prepared as is known in the art (e.g., as described in U.S. Pat. No. 9,382,266 B2) and purified by any suitable separation technology including, but not limited to, preparative-scale chromatography.

In some embodiments, then, this disclosure provides a compound of Formula I as described above, and/or a pharmaceutically acceptable salt, solvate, ester, or mixture thereof for use in treating and/or preventing a neurodegenerative condition. In some embodiments, this disclosure provides a composition comprising such a compound, pharmaceutically acceptable salt, solvate, ester, or mixture and, optionally, at least one pharmaceutically acceptable carrier. In some embodiments, this disclosure provides for such use of a composition comprising about 70% or more, about 80% or more, about 90% or more (i.e., “substantially pure”), about 95% or more, or about 99% or more of a compound of Formula I, a pharmaceutically acceptable salt thereof, a solvate thereof, an ester thereof, and/or mixtures of the foregoing. In some embodiments, the composition for such use contains an essentially pure mixture, wherein an essentially pure mixture may contain trace amounts or pharmaceutically insignificant amounts of other polyketides, of the compound of Formula I, pharmaceutically acceptable salts, solvates, and esters thereof, and mixtures of the foregoing. In some embodiments, this disclosure provides for the use a pharmaceutical composition comprising a pharmaceutically acceptable carrier, diluent or excipient and a polyketide of Formula I, wherein the pharmaceutically acceptable salt, solvate, and/or hydrate of the compound of Formula I comprises at least about 70%, at least about 80%, at least about 90%, at least about 95%, or at least about 98% of the polyketide component of the pharmaceutical composition in treating and/or preventing a neurodegenerative condition. In some embodiments of such uses, that polyketide compound is essentially the only polyketide in the pharmaceutical composition. In some embodiments, the solvate, if present, is a hydrate.

In some embodiments, this disclosure also provides methods for treating a mammal in need of prevention and/or treatment of a neurodegenerative disease, the method comprising administering to said mammal (e.g., a human being) an effective amount (e.g., a therapeutically effective amount) of the compound, pharmaceutically acceptable salt thereof, a solvate thereof, an ester thereof of the compound of Formula I, and/or a composition and/or mixture comprising the same. In some embodiments, the method of treating a mammal in need thereof comprises administering to said mammal an effective amount of a compound of Formula I, pharmaceutically acceptable salt thereof, solvate thereof, ester thereof, or mixture thereof and/or comprising the compound of Formula I, and/or a composition comprising Formula I (e.g., in a therapeutically effective amount) thereto. In some embodiments, the mammal has a disease selected from the group consisting of but are not limited to, for Alzheimer's disease, multiple sclerosis, Parkinson's disease, and/or Huntington's disease.

In some embodiments, the method comprises administration of the compound of Formula I to a mammal at about any of 1, 2, 3, 4, 5, 6, 7, 8, 9, 10, 11, 12, 13, 14, 15, 16, 17, 18, 19, or 20 mg/kg (e.g., about 10 mg/kg) to provide an effective mean concentration of in the whole blood of the mammal for a sufficient period of time (e.g., up to six hours) following administration. In some embodiments, the method comprises administering the compound of Formula I to the mammal multiple times in a single day and/or over several days, weeks, months, and/or years. In some embodiments, such administration results in a reduction in the symptoms of the neurodegenerative condition to, e.g., about any of 20%, 25%, 30%, 40%, 50%, 60%, 70%, 80%, or 90% of symptoms before treatment was started, or between any two points in time and/or doses. In some embodiments, a significant reduction in symptoms is observed and exhibits an adjusted P value of 0.0001 as determined by Dunnett's multiple comparison's test.

Any mode of administration may be utilized. In some embodiments, the compound, composition and/or mixture is administered by application to an implantable medical device (e.g., a stent).

In some embodiments, then, this disclosure provides methods for preventing and/or treating a neurodegenerative condition in a mammal by administering a compound of Formula I, a pharmaceutically acceptable salt, solvate, ester, or mixture thereof in a therapeutically effective amount, and/or a composition comprising the same. In some embodiments, the neurodegenerative condition includes but is not limited to Alper's disease, Alzheimer's disease, amyotrophic lateral sclerosis (ALS), motor neurone disease (MND), Lou Gehrig's Disease, Batten disease, choreic syndrome, dystonic syndrome, Friedrich's ataxia, Huntington's disease, Lewy body disease, primary or secondary Parkinson's disease, prion disease, spinocerebellar ataxia, spinal muscular dystrophy, aging, alcoholism, cancer, injury, stroke, infectious disease, psychological disorders, and toxicity. In some embodiments, the neurodegenerative condition is Alzheimer's disease, multiple sclerosis, Parkinson's disease, or Huntington's disease. In some embodiments, the compound of Formula I, or compositions comprising the same, is administered as the sole active pharmaceutical agent. In some embodiments, the compound of Formula I, or a composition comprising the same, is administered in combination with one or more agents selected from the group consisting of analgesics, anti-amyloidogenic, anti-cancer agents, anti-cholinergic agents, anti-convulsives, anti-depressants, anti-oxidants, anti-psychotics, anxiolytics, cell cycle inhibitors, dopamine releasing agents, dopamine replenishing agents, dopaminergic agonists, immunomodulatory, anti-inflammatory, immunostimulant, metabolic modulator, neuroprotectants, nootropic agents, vasoprotectants, and vasodilatory agents. In some embodiments, the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately. In some embodiments, the compound of Formula I is provided as a bead, tablet, capsule, solution, or suspension. Also provided are uses of a compound of Formula I in the preparation of a medicament for the treatment of a neurodegenerative condition (e.g., as described above). In some embodiments, kits for treating a patient having a neurodegenerative condition, the kit comprising at least one therapeutically effective dose of the compound of Formula I, and instructions for preventing and/or treating a neurodegenerative condition using the same are also provided. In some embodiments, the instructions refer to administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately. In some embodiments, the kit provides the compound of Formula I as a bead, tablet, capsule, solution, or suspension. Other embodiments are also contemplated as would be understood by those of skill in the art.

When the terms treat, prevent, and/or ameliorate or derivatives thereof are used herein in connection with a given treatment for a given condition, it is meant to convey that the treated patient either does not develop a clinically observable level of the condition at all, or develops it more slowly and/or to a lesser degree than he/she would have had absent the treatment. These terms are not limited solely to a situation in which the patient experiences no symptom of the condition whatsoever. For example, a treatment will be said to have prevented the condition if it is given during exposure of a patient to a stimulus that would have been expected to produce a given manifestation of the condition, and results in the patient's experiencing fewer and/or milder symptoms of the condition than otherwise expected. For instance, a treatment can “prevent” symptoms where the mammal expresses less symptoms than would have been observed in the absence of treatment; it does not imply that the mammal must not exhibit any symptoms. Similarly, reduce, reducing, and reduction as used herein in connection with prevention, treatment and/or amelioration of a given condition by a particular treatment typically refers to a subject developing an infection more slowly or to a lesser degree as compared to a control or basal level (e.g., of symptoms) in the absence of a treatment.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. As used herein, a subject or a host or a mammal is meant to be an individual. The subject can include mammals such as domesticated animals, such as cats and dogs, livestock (e.g., cattle, horses, pigs, sheep, and goats), laboratory animals (e.g., mice, rabbits, rats, guinea pigs) and birds. A mammal may also be a primate or a human. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different compounds or molecules or may not include a combination such that the description includes both the combination and the absence of the combination (i.e., individual members of the combination). The term “combined” or “in combination” or “in conjunction” may refer to a physical combination of agents that are administered together or the use of two or more agents in a regimen (e.g., administered separately, physically and/or in time) for treating, preventing and/or ameliorating a particular disease. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. All references referred to in this application, including patent and patent applications, are incorporated herein by reference into this disclosure in their entirety.

Certain embodiments are further described in the following examples. These embodiments are provided as examples only and are not intended to limit the scope of the claims in any way.

### EXAMPLES

### Example 1

This example illustrates one method for determining the pharmacokinetics and bioavailability of the compound of Formula I.

A person of skill in the art will be able to determine the pharmacokinetics and bioavailability of the compound of Formula I using in vivo and in vitro methods known to a person of skill in the art, including but not limited to those described below and in Gallant-Haidner et al, 2000 and Trepanier et al, 1998 and references therein. The bioavailability of a compound is determined by a number of factors, (e.g. water solubility, cell membrane permeability, the extent of protein binding and metabolism and stability) each of which may be determined by in vitro tests as described in the examples herein, it will be appreciated by a person of skill in the art that an improvement in one or more of these factors will lead to an improvement in the bioavailability of a compound. Alternatively, the bioavailability of the compound of Formula I may be measured using in vivo methods as described in more detail below, or in the examples herein.

In order to measure bioavailability in vivo, a compound of Formula I may be administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below.

For example, mice or rats are dosed with 1 or 3 mg/kg of the compound of a compound for Formula I intravenously (i.v.) or 1, 5 or 10 mg/kg of the compound of Formula I p.o. Blood samples are taken at 5 min, 15 min, 1 h, 4 h and 24 h intervals, and the concentration of the compound of Formula I (or a metabolite thereof) in the sample is determined via LCMS-MS. The time-course of plasma or whole blood concentrations can then be used to derive key parameters such as the area under the plasma or blood concentration-time curve (AUC—which is directly proportional to the total amount of unchanged drug that reaches the systemic circulation), the maximum (peak) plasma or blood drug concentration, the time at which maximum plasma or blood drug concentration occurs (peak time), additional factors which are used in the accurate determination of bioavailability include: the compound's terminal half-life, total body clearance, steady-state volume of distribution and F %. These parameters are then analyzed by non-compartmental or compartmental methods to give a calculated percentage bioavailability, for an example of this type of method see Gallant-Haidner et al, 2000 and Trepanier et al, 1998, and references therein. Bioavailability may be determined as described herein in order to carry out studies of the effects of the compounds of Formula I in neurodegenerative disease models and/or humans.

Shown below are whole blood concentrations following administration of the compound of Formula I to mice. The compound of Formula I was administered to mice at 2 mg/kg or 10 mg/kg for three days and the concentration of the compound in whole blood determined. These determinations were made using protein precipitation, liquid chromatography (LC), and mass spectrometry (MS/MS). Ten μl aliquots of whole blood and matrix calibration standards were distributed in a 96-well plate; 10 μl aliquots of blank matrix for matrix blanks and control blanks were included as controls. Ten μl of water was added to each sample followed by vortexing. One hundred sixty ml of internal standard was added to each sample except the matrix blanks; 160 μl 70:30 water:acetonitrile (ACN) was added to matrix blanks. This was followed by a five-minute vortex at >3500 rpm. One hundred fifty μl of the resultant supernatant was then transferred to a new 96-well plate and the samples blown to dryness at 35° C. The resultant product was then reconstituted with 90 μl ACN. LC was carried out using the equipment, conditions and calibration standards are shown in Tables 1 and 2.

TABLE 1

| LC Conditions: | | | | |
|---|---|---|---|---|
| Column Id. & | Waters HSS T3, | Time | % | Flow |
| Dimensions: | 30 × 2.1 mm | (sec) | MPB | (mL/min) |
| Temperature (° C.) | Ambient | 15 | 65 | 0.800 |
| Mobile Phase A | 0.1% Formic Acid in Water | 75 | 85 | 0.800 |
| Mobile Phase B | 0.1% Formic Acid in Acetonitrile | 5 | 95 | 0.800 |
| Needle Rinse 1 | 25:25:25:25:0.1 | 25 | 95 | 0.800 |
| | MeOH:H2O:ACN:IPA:NH4OH | | | |
| Needle Rinse 2 | 10:90:0.1 MeOH:H2O:FA | 30 | 65 | 0.800 |

| | |
|---|---|
| MS Conditions | |
| MS/MS: | API-5500 |
| Ionization Method: | Electrospray |
| Positive/Negative Ion: | Positive |
| Resolution: | Unit |
| Source Temperature (° C.): | 550 |
| Transitions (m/z): | Formula I: 918.5/409.3 Da//Int Std |
| | d4-AEA: 352.1/66.0 Da |

TABLE 2
Summary of Calibration Standard Back-Calculated
Concentrations of Formula I in Mouse Whole Blood

| | STD | | | | | | | | | | |
|---|---|---|---|---|---|---|---|---|---|---|---|
| | 1 | 2 | 3 | 4 | 5 | 6 | 7 | 8 | 9 | 10 | 11 |
| | Nominal Concentration (ng/mL) | | | | | | | | | | |
| | 1.00 | 2.00 | 5.00 | 10.0 | 25.0 | 50.0 | 100 | 200 | 500 | 800 | 1000 |
| | NR | NR | NR | 12.4 | 21.0 | 47.6 | 115 | 237 | 580 | 795 | 993 |
| | NR | NR | NR | 9.21 | 20.4 | 40.4 | 95.7 | 208 | 484 | 757 | 978 |
| Mean | NA | NA | NA | 10.8 | 20.7 | 44.0 | 105.4 | 223 | 532 | 776 | 986 |
| SD | NA | NA | NA | 2.26 | 0.424 | 5.09 | 13.6 | 20.5 | 67.9 | 26.9 | 10.61 |
| % Bias | NA | NA | NA | 8.1 | −17.2 | −12.0 | 5.3 | 11.3 | 6.4 | −3.0 | −1.5 |
| n | 0 | 0 | 0 | 2 | 2 | 2 | 2 | 2 | 2 | 2 | 2 |

N/A = Not Applicable // NR = Not Reported

The results of these analyses are summarized in Tables 3 and 4.

TABLE 3

| Formula I | Formula 1 Concentrations (ng/mL) in Mouse Whole Blood | | | | | |
|---|---|---|---|---|---|---|
| (2 mg/kg) | | | | | | |
| Time Points | Animal ID | | | | | |
| (hrs) | 1 | 2 | 3 | Mean | SD | % CV |
| IP (QD x | | | | | | |
| 3 days)- | | | | | | |
| Day 1 | | | | | | |
| 1.00 | 519 | 530 | 537 | 529 | 9.07 | 1.7% |
| 2.00 | 668 | 392 | 233 | 431 | 220 | 51.1% |
| 6.00 | 468 | 646 | 344 | 486 | 152 | 31.2% |
| 24.0 | 35.8 | 48.0 | 26.0 | 36.6 | 11.0 | 30.1% |
| Day 2 | | | | | | |
| 1.00 | 682 | 670 | 354 | 569 | 186 | 32.7% |
| 2.00 | 746 | 841 | 854 | 814 | 59.0 | 7.2% |
| 6.00 | 537 | 803 | 315 | 552 | 244 | 44.3% |
| 24.0 | 26.8 | 76.5 | 40.1 | 47.8 | 25.7 | 53.8% |
| Day 3 | | | | | | |
| 1.00 | 968 | 885 | 986 | 946 | 53.9 | 5.7% |
| 2.00 | 760 | 707 | 282 | 583 | 262 | 44.9% |
| 6.00 | 560 | 860 | 469 | 630 | 205 | 32.5% |
| 24.0 | 45.7 | 92.2 | 49.7 | 62.5 | 25.8 | 41.2% |

TABLE 4

| Formula I | Formula I Concentrations (ng/mL) in Mouse Whole Blood | | | | | |
|---|---|---|---|---|---|---|
| (10 mg/kg) | | | | | | |
| Time Points | Animal ID | | | | | |
| (hrs) | 4 | 5 | 6 | Mean | SD | % CV |
| IP (QD x | | | | | | |
| 3 days)- | | | | | | |
| Day 1 | | | | | | |
| 1.00 | 744 | 736 | 853 | 778 | 65.4 | 8.4% |
| 2.00 | 2390 | 2040 | 2590 | 2340 | 278 | 11.9% |
| 6.00 | 1970 | 2300 | 908 | 1726 | 727 | 42.1% |
| 24.0 | 357 | 320 | 141 | 273 | 116 | 42.4% |
| Day 2 | | | | | | |
| 1.00 | AQL | 3130 | 739 | 1935 | NA | NA |
| 2.00 | 3060 | 2660 | 2740 | 2820 | 211.7 | 7.5% |
| 6.00 | AQL | AQL | 811 | 811 | NA | NA |
| 24.0 | 335 | 472 | 199 | 335 | 137 | 40.7% |
| Day 3 | | | | | | |
| 1.00 | 2890 | 3490 | 3220 | 3200 | 300 | 9.4% |
| 2.00 | 2530 | 2680 | 482 | 1897 | 1228 | 64.7% |
| 6.00 | 966 | 2410 | 773 | 1383 | 895 | 64.7% |
| 24.0 | 292 | 548 | 276 | 372 | 153 | 41.0% |

AQL: Original value above quantitation limit (1000 ng/mL). Sample was diluted prior to re-analysis, but due to a sequence error the samples will be re-injected.

As shown above, Formula I exhibits sufficient concentrations in whole blood over time following administration at 2 mg/kg or 10 mg/kg via the intraperitoneal route (IP) once daily (QD) for three days. To carry out these assays using brain homogenates, 20 uL of samples and matrix calibration standards into wells of a 96-well plate, and 10 uL of blank matrix allocated for matrix blanks and control blanks. 160 uL internal standard was then added to all samples except matrix blanks, and 160 uL ACN added to matrix blanks. Samples were then vortexed for 5 minutes and centrifuge for 5 minutes at >3500 rpm. 150 uL of the supernatant was then transferred to a new 96-well plate, and the plates blown down to dryness at 35° C., reconstituted with 90 uL of 70:30 water:acetonitrile (ACN) solution, covered and vortexed prior to analysis. LC and MS conditions were the same as described above for the whole blood analysis. A summary of the Calibration of Standard Back-Calculated Concentration of Formula I in brain homogenate is shown in Table 5.

TABLE 5

| | STD | | | | | | | | | | |
|---|---|---|---|---|---|---|---|---|---|---|---|
| | 1 | 2 | 3 | 4 | 5 | 6 | 7 | 8 | 9 | 10 | 11 |
| | Nominal Concentration (ng/mL) | | | | | | | | | | |
| | 0.500 | 1.00 | 2.00 | 5.00 | 10.0 | 50.0 | 100 | 200 | 500 | 800 | 1000 |
| | NR | NR | NR | 4.36 | 10.9 | 59.3 | 95.9 | 166 | 575 | 675 | 892 |
| | NR | NR | NR | 4.92 | 11.3 | 62.5 | 105 | 172 | 566 | 672 | 939 |
| Mean | NA | NA | NA | 4.64 | 11.1 | 60.9 | 100 | 169 | 571 | 674 | 916 |
| SD | NA | NA | NA | 0.396 | 0.283 | NA | 6.43 | 4.24 | 6.36 | 2.12 | 33.2 |
| % Bias | NA | NA | NA | −7.2 | 11.0 | 21.8 | 0.5 | −15.5 | 14.1 | −15.8 | −8.5 |
| n | 0 | 0 | 0 | 2 | 2 | 2 | 2 | 2 | 2 | 2 | 2 |

NA = not applicable;
NR = not reported

Shown below are brain concentrations following administration of the compound of Formula I to mice. The compound of Formula I was administered to mice at 2 mg/kg or 10 mg/kg for three days and the concentration of the compound in brain determined. The results are summarized in Table 6:

TABLE 6

| IP (QD x 3 days)- | Formula I Concentrations (ng/g) in Mouse Brain | | | | | |
|---|---|---|---|---|---|---|
| Day 3 | Animal ID | | | | | |
| Time Points (hrs) | 1 | 2 | 3 | Mean | SD | % CV |
| Group 1 (2 mg/kg) | | | | | | |
| 24.0 | 31.1 | 39.2 | 68.1 | 46.1 | 19.5 | 42.2% |
| Group 2 (10 mg/kg) | | | | | | |
| 24.0 | 140 | 140 | 299 | 193 | 91.8 | 47.6% |

As shown above, Formula I exhibits sufficient concentrations in brain over time following administration at 2 mg/kg or 10 mg/kg via the intraperitoneal route (IP) once daily (QD) for three days.

### Example 2

The efficacy of the compound of Formula I may be tested in in vivo models for neurodegenerative conditions (e.g., a neurodegenerative disease) which are described herein and which are known to a person of skill in the art. Such models include, but are not limited to, for Alzheimer's disease—animals that express human familial Alzheimer's disease (FAD) p-amyloid precursor (APP), animals that overexpress human wild-type APP, animals that overexpress p-amyloid 1-42 (pA), animals that express FAD presenillin-1 (PS-1) (e. g. German and Eisch, 2004). For multiple sclerosis—the experimental autoimmune encephalomyelitis (EAE) model (see Bradl, 2003). For Parkinson's disease—the 1-methyl-4-phenyl-1,2,3,6-tetrahydropyridine (MPTP) model or the 6-hydroxydopamine (6-OHDA) model (see e.g. Emborg, 2004; Schober A. 2004). For Huntington's disease there are several models including the R6 lines model generated by the introduction of exon 1 of the human Huntington's disease (HD) gene carrying highly expanded CAG repeats into the mouse germ line (Sathasivam et al, 1999) and others (see Hersch and Ferrante, 2004). A 6-OHDA Parkinson's disease model was used to determine the effectiveness of the compound of Formula I in treating neurodegenerative conditions.

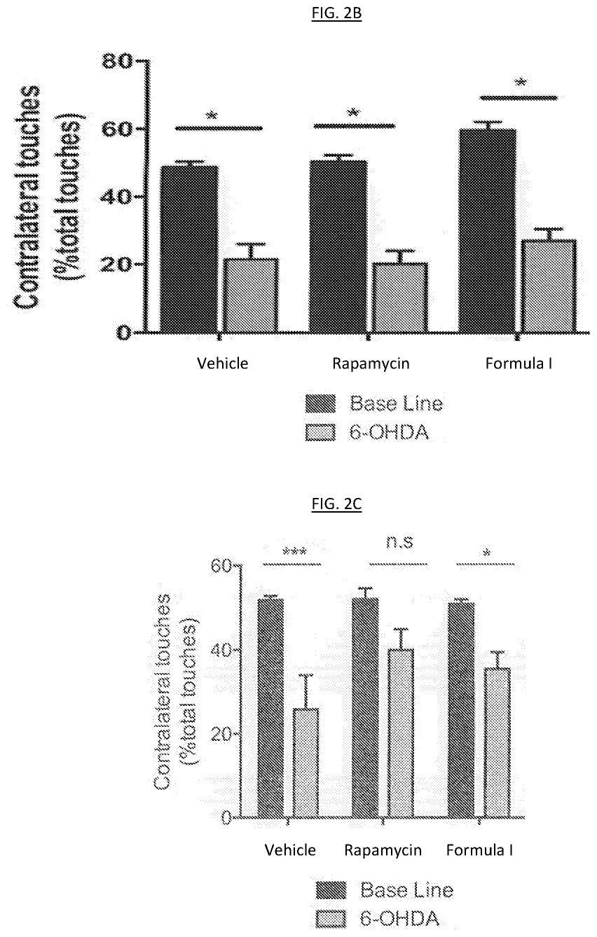

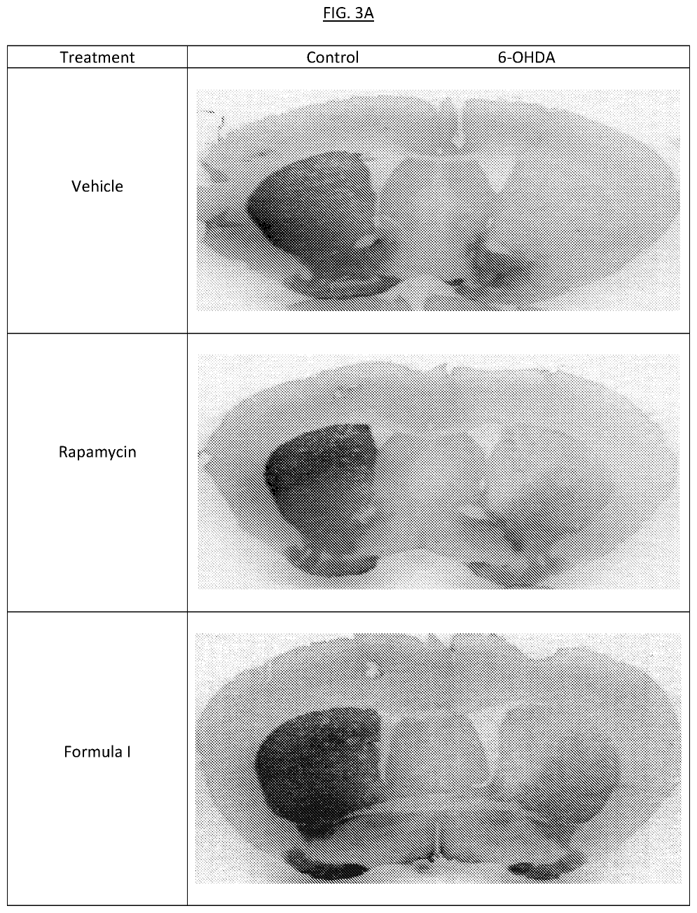

An analysis of the neuroprotective effects of the compound of Formula I on motor performance was performed using the 6-OHDA cylinder test (FIG. 2A) which evaluates spontaneous motor changes associated with dopamine depletion in the striatum of 6-OHDA injected mice. Mice were pre-treated with the compound of Formula I (in 2% EtOH, 40% PEG 400 in saline (PBS 1×)) at a dose of 10 mg/kg, rapamycin (10 mg/kg) (in 2% EtOH, 40% PEG 400 in saline (PBS 1×)), or the vehicle control (2% EtOH, 40% PEG 400 in saline (PBS 1×)) for three days prior to stereotactic injection of 6-OHDA (80 μg) into one hemisphere of the brain. 6-OHDA-treated mice were placed in a glass container and the number times each mouse touched the glass wall with each forepaw following administration of vehicle only (n=7) (control mice), rapamycin (n=10), or the compound of Formula I (n=10) was measured. “Baseline” mice were treated with 6-OHDA, vehicle, rapamycin or the compound of Formula I after the baseline measurement was taken. The data presented in FIG. 2B as mean and standard error of the mean (SEM), for a seven day test. Statistical analyses as compared to baseline mice were performed using the one-way Anova test (*: p<0.05). As shown therein, the compound of Formula I outperformed rapamycin and the vehicle control by bringing the number of touches closer to that of baseline mice. The data presented in FIG. 2C was generated in a similar manner as was performed to generate the data presented in FIG. 2B (except that Formula I or rapamycin was administered at 3 mg/kg and pre-treatment prior to the first 6-OHDA administration was for two days), and supports the data presented in FIG. 2B (vehicle (n=6); rapamycin (n=7); Formula I (n=8)). Data is presented as mean and SEM. Statistical analyses were performed using one-way Anova test (*: p<0.05; ***: p<0.001. n.s.: not significant.)

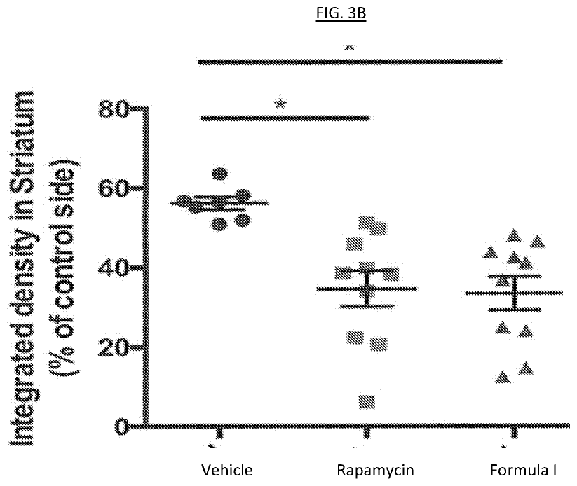

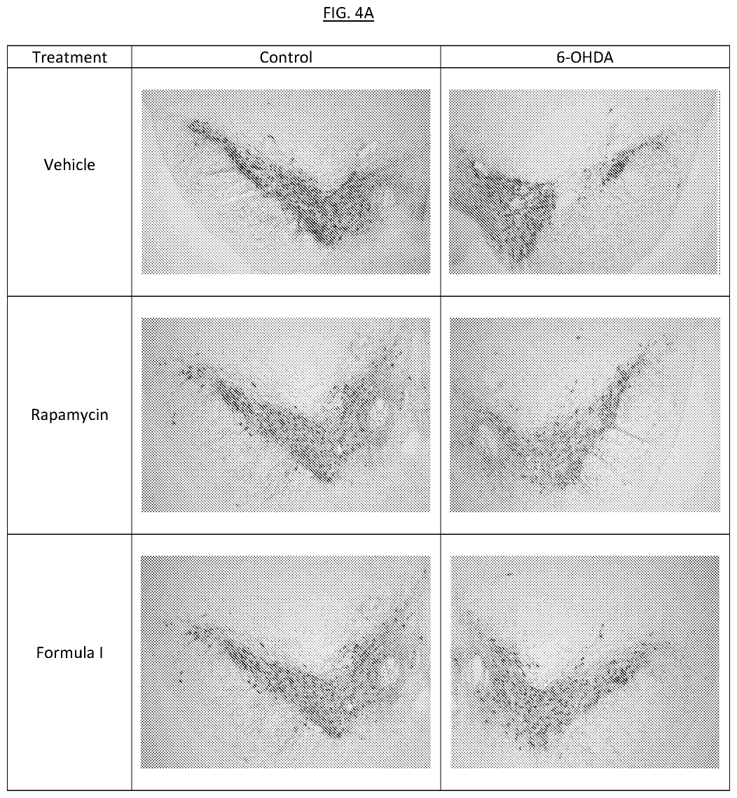

Immunohistochemistry analysis using anti-tyrosine hydroxylase (TH) antibody was performed in mouse midbrain sections to quantify 6-OHDA-induced striatal denervation in mice injected with 6-OHDA and non-injected (control) sides of the brain. The integrated density of pixel intensity of TH stain covering the entire striatum and expressed as a percentage of TH loss relative to the control slide, thereby determining the effect of vehicle control (treatment negative control (n=7)), rapamycin (n=10) and the compound of Formula I (n=10) on 6-OHA-induced denervation. FIG. 3A provides a representative visual evidence that rapamycin and the compound of Formula I (10 mg/kg) decreased 6-OHDA-induced denervation as compared to vehicle control. FIG. 3A also provides representative visual evidence that the compound of Formula I also decreased 6-OHDA-induced denervation as compared to rapamycin. For instance, in the Vehicle panels, the 6-OHDA/vehicle-treated striatum side shows essentially no to very little anti-TH antibody staining. In contrast, both the 6-OHDA/rapamycin-treated and the 6-OHDA/Formula I-treated sides of the brain show increased staining with the anti-TH antibody, as indicated by the dark shading in those figures. FIG. 3B presents the mean and standard error of the mean (SEM) of the integrated density of anti-TH antibody staining in the striatum, as well as statistical analysis of the results for the groups of mice. As shown therein, the one-way ANOVA test showed a significant difference between vehicle control and rapamaycin (p<0.05) as well as between vehicle control and the compound of Formula I (p<0.05). Accordingly, the compound of Formula I was shown to protect mice from 6-OHDA-induced striatal denervation as compared to vehicle control as well as rapamycin.

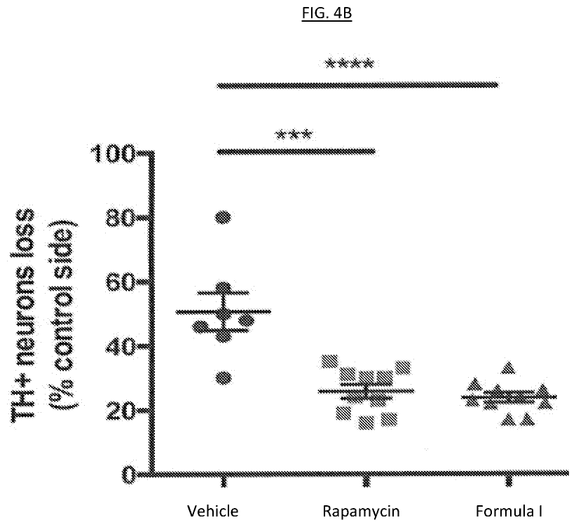

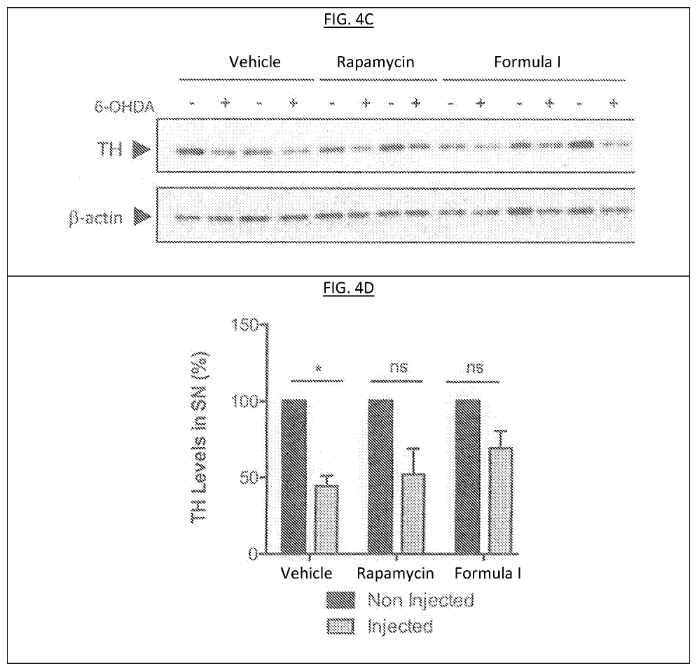

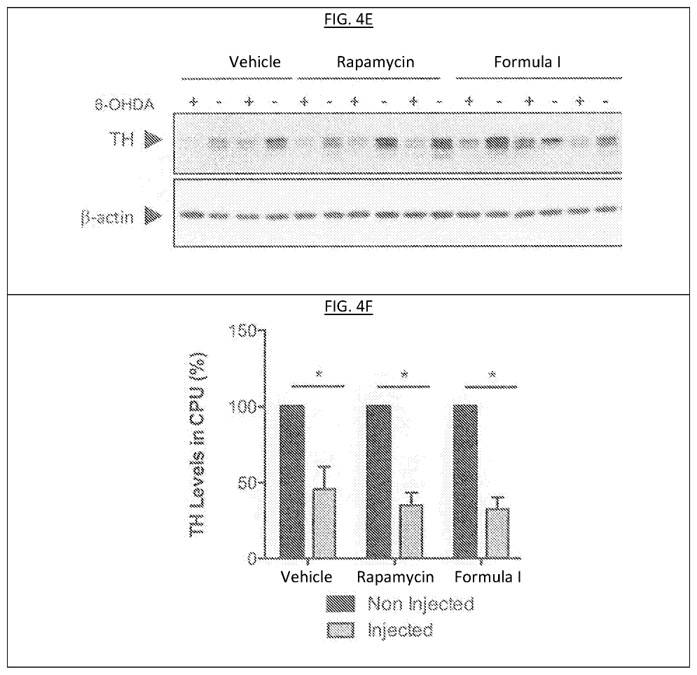

Immunohistochemistry analysis using anti-tyrosine hydroxylase (TH) antibody was also performed to quantify 6-OHDA-induced neuronal loss in mice injected with 6-OHDA and non-injected (control) sides of the brain. The total content of TH-positive somas was measured in midbrain sections covering the entire SN in the 6-OHDA injected and non-injected sides for each group of mice, and the effect of vehicle control (treatment negative control (n=7)), rapamycin (n=10) and the compound of Formula I (n=10) (10 mg/kg) thereupon. FIG. 4A provides a representative visual evidence that rapamycin and the compound of Formula I decreased 6-OHDA-induced neuronal loss as compared to vehicle control. FIG. 4B presents the mean and standard error of the mean (SEM) as well as statistical analysis of the results. As shown therein, the one-way ANOVA test showed a significant difference between vehicle control and rapamaycin (p<0.001) as well as between vehicle control and the compound of Formula I (p<0.0001). This staining data was confirmed by staining for TH protein expression in the 6-OHDA-treated striatum. As shown in FIGS. 4C, 4D, 4E and 4F (3 mg/kg dose of Formula I or rapamycin, two-day pre-treatment), TH protein expression in 6-OHDA-treated striatum (“Injected” with 6-OHDA) showed a statistically significant difference from control striatum (“Not Injected” with 6-OHDA), but non-statistically significant difference from 6-OHDA/rapamycin-treated (“Injected” with 6-OHDA) or 6-OHDA/Formula I-treated (“Injected” with 6-OHDA) striatum. Accordingly, the compound of Formula I was shown to protect mice from 6-OHDA-induced neuronal loss.

In sum, this data shows that the compound of Formula I provides neuroprotection from 6-OHDA-induced neurotoxicity. As the 6-OHDA model is generally accepted as representative of Parkinson's disease, those of skill in the art would understand the compound of Formula I to have anti-Parkinson's disease effects, i.e., to be potentially useful in treating and/or preventing Parkinson's Disease in mammals such as human beings.

All references referred to in this application, including patent and patent applications, are incorporated herein by reference into this disclosure in their entirety. Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

What is claimed is:

1. A method for preventing and/or treating a neurodegenerative condition in a mammal, the method comprising administering to the mammal a compound of Formula I: 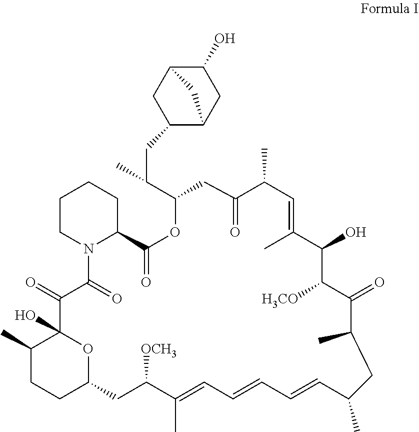 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2C[C@@H]3C[C@H]2C[C@H]3O)CC(=O)[C@H](C)/C=C(\C)[C@@H](O)[C@@H](OC)C(=O)[C@H](C)C[C@H](C)/C=C/C=C/C=C/1C]
and/or,
a pharmaceutically acceptable salt, solvate, ester, or mixture thereof.

2. The method of claim 1 wherein the neurodegenerative condition is selected from the group consisting of Alper's disease, Alzheimer's disease, amyotrophic lateral sclerosis (ALS), motor neurone disease (MND), Lou Gehrig's Disease, Batten disease, choreic syndrome, dystonic syndrome, Friedrich's ataxia, Huntington's disease, Lewy body disease, primary or secondary Parkinson's disease, prion disease, spinocerebellar ataxia, spinal muscular dystrophy, aging, alcoholism, cancer, injury, stroke, infectious disease, psychological disorders, and toxicity.

3. The method of claim 3 wherein the neurodegenerative condition is selected from the group consisting of Alzheimer's disease, multiple sclerosis, Parkinson's disease, and Huntington's disease.

4. The method of any one of claims 1-3 wherein the compound of Formula I is administered as the sole active pharmaceutical agent; or the compound of Formula I is administered in combination with one or more agents selected from the group consisting of analgesics, anti-amyloidogenic, anti-cancer agents, anti-cholinergic agents, anti-convulsives, anti-depressants, anti-oxidants, anti-psychotics, anxiolytics, cell cycle inhibitors, dopamine releasing agents, dopamine replenishing agents, dopaminergic agonists, immunomodulatory, anti-inflammatory, immunostimulant, metabolic modulator, neuroprotectants, nootropic agents, vasoprotectants, and vasodilatory agents.

5. The method of any one of claims 1-4 wherein the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

6. The method of any one of claims 1-5 wherein the compound of Formula I is provided as a bead, tablet, capsule, solution, or suspension.

7. Use of a compound of Formula I in the preparation of a medicament for the prevention and/or treatment of a neurodegenerative condition.

8. The use of claim 7 wherein the neurodegenerative condition is selected from the group consisting of Alper's disease, Alzheimer's disease, amyotrophic lateral sclerosis (ALS), motor neurone disease (MND), Lou Gehrig's Disease, Batten disease, choreic syndrome, dystonic syndrome, Friedrich's ataxia, Huntington's disease, Lewy body disease, primary or secondary Parkinson's disease, prion disease, spinocerebellar ataxia, spinal muscular dystrophy, aging, alcoholism, cancer, injury, stroke, infectious disease, psychological disorders, and toxicity.

9. The use of claim 7 or 8 wherein the compound of Formula I is administered as the sole active pharmaceutical agent; or the compound of Formula I is administered in combination with one or more agents selected from the group consisting of analgesics, anti-amyloidogenic, anti-cancer agents, anti-cholinergic agents, anti-convulsives, anti-depressants, anti-oxidants, anti-psychotics, anxiolytics, cell cycle inhibitors, dopamine releasing agents, dopamine replenishing agents, dopaminergic agonists, immunomodulatory, anti-inflammatory, immunostimulant, metabolic modulator, neuroprotectants, nootropic agents, vasoprotectants, and vasodilatory agents

10. The use of any one of claims 7-9 wherein the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

11. The use of any one of claims 7-10 wherein the compound of Formula I is provided as a bead, tablet, capsule, solution, or suspension.

12. A kit for preventing and/or treating a neurodegenerative condition, the kit comprising at least one therapeutically effective dose of the compound of Formula I, and instructions for preventing and/or treating a neurodegenerative condition using the same.

13. The kit of claim 10 wherein the instructions refer to administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

14. The kit of claim 12 or 13 wherein the compound of Formula I is provided as a bead, tablet, capsule, solution, or suspension.
